(19) United States
(12) Patent Application Publication
(10) Pub. No.: US 2020/0060883 A1
(43) Pub. Date: Feb. 27, 2020

(54) Tampon

(71) Applicant: Ramón Vendrell Vila, Balsareny, Barcelona (ES)

(72) Inventor: Ramón Font Caselles, Manresa, Barcelona (ES)

(21) Appl. No.: 16/308,869

(22) PCT Filed: Jun. 20, 2017
(86) PCT No.: PCT/ES2017/000074

§ 371 Date: Dec. 11, 2018

(30) Foreign Application Priority Data
Jun. 20, 2016 (ES) U-201600438

(51) Int. Cl. A61F 13/20; A61F 13/551; A61F 13/26; A61F 13/34

(52) U.S. Cl. CPC A61F 13/206; A61F 13/55175; A61F 13/2065; A61F 13/34; A61F 13/266

(57) ABSTRACT

A tampon, particularly for feminine hygiene, comprises a substantially cylindrical absorbent body that is peripherally compressed and contained in a wrapper or in an applicator device. The absorbent body is formed by a rolled rectangular strip of absorbent material and comprises: a front end in the direction of insertion of the tampon, a rear end from which extends a removal string, and a lateral surface. The entirety of the outer surface of the absorbent body, corresponding to the front end, rear end and lateral surface is formed by a first layer of non-woven absorbent fabric.

### FIELD OF THE INVENTION

This invention is applicable in the field dedicated to the manufacture of products for intimate feminine hygiene and more particularly, to a tampon for feminine hygiene.

### BACKGROUND

There are currently in the market several tampons for feminine hygiene that are generally comprised of an absorbent body with a cylindrical configuration which is compressed inside an applicator device or in a wrapper, said absorbent body being formed by a rolled strip of absorbent material that comprises an absorbent core, which includes absorbent fibers.

These tampons generally have the drawback that the absorbent fibers in the core are arranged on the exterior of the absorbent body of the tampon.

This external arrangement of the absorbent fibers means that when the tampon is being used, the fibers are unprotected and remain in contact with the vagina of the user, causing irritation, particularly in the final days of menstruation, since said absorbent fibers adhere to the user due to the lower amount of discharge, drying out and making it difficult to remove the tampon.

In the tampons currently in the market, the absorbent fibers are exposed in the entirety of the lateral surface of the cylindrical body and/or in the front and rear ends of the tampon, causing the aforementioned problems.

The applicant of the invention is unaware of the existence of tampons with the suitable characteristics required to address these drawbacks.

### DESCRIPTION OF THE INVENTION

The object of the invention is a tampon, particularly for feminine hygiene; which comprises a substantially cylindrical absorbent body that is peripherally compressed and contained in a wrapper or in an applicator device, with an absorbent body formed by a rolled rectangular strip of absorbent material, and said absorbent body comprises: a front end in the direction of insertion of the tampon, a rear end from which extends a removal string and a lateral or peripheral surface.

This tampon has a series of characteristics designed to address the technical problem mentioned above; and more specifically to prevent the absorbent fibers from coming into contact with the body of the user and therefore adhering to the body, causing irritation or making it difficult to remove the tampon.

In order to achieve this and according to the invention, in the solution provided, the entirety of the outer surface of the absorbent body corresponding to the front end, the rear end and the lateral surface thereof, are formed of a first layer of non-woven absorbent fabric.

In this first layer of non-woven fabric, the fibers comprising the fabric are joined together by means of punching, for example, preventing them from coming loose and adhering to the body of the user, causing irritation or making it difficult to remove the tampon.

In a first embodiment of this invention the strip of absorbent material is formed by the aforementioned first layer of non-woven absorbent fabric. In this embodiment the absorbent body lacks an absorbent core, which is intended for those cases in which tampons are manufactured with a low absorption capacity.

In this embodiment the first layer of non-woven fabric comprises: a central longitudinal portion of a width equal to the length of the tampon, with an outer side and an inner side in relation to the direction in which it is rolled up; and two lateral longitudinal wings folded over the inner side of the central longitudinal portion.

In a second embodiment of the invention, the strip of absorbent material also comprises an absorbent core that can have a variable thickness depending on the desired absorption capacity of the tampon.

This absorbent core, which is formed by absorbent fibers such as cotton, viscose or similar, is located between the central portion and the folded wings of the first layer of non-woven fabric; with the outer side and longitudinal edges thereof being completely covered by said first layer of non-woven fabric; and at least two lateral strips of the inner side thereof being covered by the wings of the aforementioned first layer of non-woven fabric.

In this way, when the absorbent strip of material is rolled up in order to form the body of the tampon, the absorbent core formed by the absorbent fibers is completely covered by the first layer of non-woven fabric, with said first layer preventing the absorbent fibers of the absorbent core from adhering to the user when it is being used, and causing her irritation.

According to other characteristics of the invention, independently of whether the absorbent body comprises an absorbent core, it comprises on the exterior of the lateral surface thereof some means for holding the strip of absorbent material in the rolled position, with said means comprising mechanical punching or pressurized water jets applied on the strip of absorbent material itself after being rolled up, or by a supporting strip secured with heat, for example, on said lateral surface of the absorbent body.

### BRIEF DESCRIPTION OF THE FIGURES

To complement the description being made, and to make it easier to understand the characteristics of the invention, this descriptive report is accompanied by a set of drawings in which, for illustrative purposes only, and without limitation, the following has been represented wherein:

FIG. 1 shows a schematic perspective view of the tampon that is the object of the invention contained in an applicator device;

FIG. 2 shows a perspective view of a first example embodiment of the substantially cylindrical absorbent body of the tampon, formed by a rolled rectangular strip of absorbent material and comprising a first layer of non-woven absorbent fabric;

FIG. 3 shows a perspective view of the development of the absorbent strip comprising the body of the tampon in FIG. 2, which is provided on one of the ends thereof with a supporting strip, which is secured by means of heat; and

FIG. 4 shows a perspective view of a variation of the embodiment of the absorbent body intended to be rolled up in order to form the body of the tampon, comprising in this case an absorbent core of absorbent fibers and also provided on one of the ends thereof with a supporting strip.

### PREFERRED EMBODIMENT OF THE INVENTION

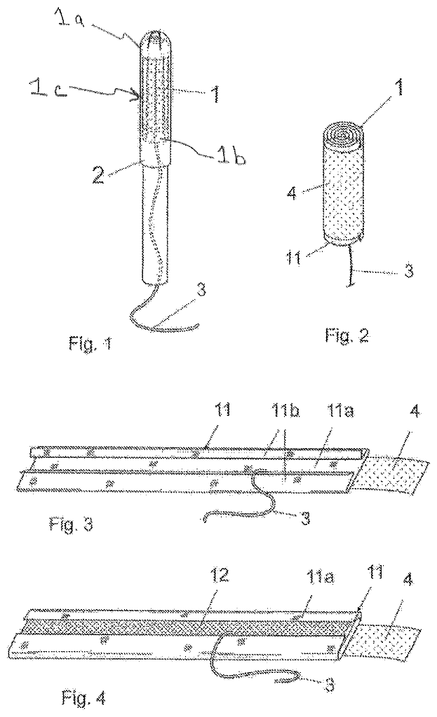

The example shown in FIG. 1 shows the tampon according to the invention, which comprises a substantially cylindrical absorbent body (1) intended to be peripherally compressed, and in this particular case to be contained in an applicator device (2).

The absorbent body (1) is formed by a rolled rectangular strip of absorbent material, and said absorbent body comprises: a front end (la) in the direction of insertion of the tampon, a rear end (1b) from which extends a removal string (3), and a lateral surface (1c).

In the example shown in FIG. 3, the strip of absorbent material intended to be rolled up in order to form the absorbent body (1) comprises a first layer (11) of non-woven fabric comprising: a central longitudinal portion (11a) of a width equal to the length of the absorbent body (1) of the tampon, with an outer side and an inner side in relation to the direction in which it is rolled up; and two lateral longitudinal wings (11b) folded over the inner side of the central portion (11a).

In this way, in the rolled position shown in FIG. 2, the entirety of the outer surface of the absorbent body (1) is formed by the aforementioned first layer (11) of non-woven fabric.

In the example shown in FIGS. 2 and 3, the strip of absorbent material comprises on one of the ends thereof a supporting strip (4), which is made of polypropylene in this case and including punching creating a number of small holes or openings in the supporting strip (4), which supporting strip (4) is secured by means of heat on the outer surface of the absorbent body (1), after it has been rolled up, preventing it from accidentally unrolling and also limiting the lateral expansion capacity thereof when the tampon is being used.

In this embodiment the absorbent body (1) is only comprised of a first layer (11) of non-woven fabric that can have a variable thickness depending on the desired absorption capacity.

In the variation of the embodiment shown in FIG. 4, the strip of absorbent material intended to form the absorbent body (1) comprises, in addition to the aforementioned first layer (11) of non-woven fabric, an absorbent core (12) that can be thicker or thinner depending on the absorption capacity of the desired tampon.

This absorbent core (12), which is formed by absorbent fibers of cotton, viscose or similar; is arranged between the central portion (11a) and the folded wings (11b) of the first layer (11) of non-woven fabric. This arrangement of the absorbent core (12) causes the outer side and longitudinal edges thereof to be entirely covered by the first layer of non-woven fabric; and also causes at least two lateral strips of the inner side thereof to be covered by the wings (11b) of the first layer (11) of non-woven fabric.

In this way, when the first layer (11) is rolled up with the absorbent core (12) in order to form a cylindrical absorbent body (1), the entirety of the outer surface of said absorbent body (1) is formed by the first layer (11) of non-woven fabric, completely hiding the fibers of the absorbent core (12), which prevents said absorbent fibers from coming into direct contact with the user, solving the problems described above.

Having described sufficiently the nature of the invention, and the examples of a preferred embodiment, it can be stated for the relevant effects that the materials, shape, size and arrangement of the elements described may be modified, provided this does not mean an alteration in the essential characteristics of the invention for which the claims are set out below as they may be amended to provide allowed claims.

1. A tampon for feminine hygiene, comprising:
a substantially cylindrical absorbent body (1) that is peripherally compressed and contained in one of a wrapper or in an applicator device (2), said absorbent body (1) having an outer surface and is formed by a rolled rectangular strip of absorbent material, wherein said absorbent body (1) comprises:
a front end (1a) in the direction of insertion of the tampon;
a rear end (1b) from which extends a removal string (3);
and a lateral surface (1c);
characterised in that the entirety of the outer surface of the absorbent body (1) corresponding to the front end (1a), rear end (1b) and lateral surface (1c) thereof is formed by a first layer (11) of non-woven absorbent fabric.

2. The tampon according to claim 1, characterised in that the rolled rectangular strip of absorbent material comprises a first layer (11) of non-woven absorbent fabric that comprises a central longitudinal portion (11a) with a width equal to the length of the absorbent body (11) of the tampon, with an outer side and an inner side in relation to the direction in which it is rolled up, and two lateral longitudinal wings (11b) folded over the inner side of the central portion (11a).

3. The tampon according to claim 2, characterised in that the strip of absorbent material comprises an absorbent core (12) having a variable thickness depending on the desired absorption capacity of the tampon, wherein said absorbent core (12) is formed by absorbent fibers, and wherein said absorbent core (12) is arranged between the central portion (11a) and the folded wings (11b) of the first layer (11) of the non-woven fabric with the outer side and longitudinal edges thereof being completely covered by said first layer (11) of non-woven fabric, and at least two lateral strips of the inner side thereof being covered by the wings (11b) of the first layer of non-woven fabric.

4. The tampon as claimed in claim 1, characterised in that the absorbent body (1) includes, on the outer surface thereof a supporting strip (4) configured for holding in place the rolled strip of absorbent material, said support strip including punching carried out in the supporting strip (4) creating a plurality of small opening.

5. The tampon as claimed in claim 4 wherein said supporting strip (4) is secured on said lateral surface (1c) of said absorbent body (1).
